(19) United States
(12) Patent Application Publication
(10) Pub. No.: US 2024/0407686 A1
(43) Pub. Date: Dec. 12, 2024

(54) Artifical Intelligence Based Remote Emotion Detection and Control System and Method

(71) Applicant: MAXIS HEALTH LLC, Edison, NJ (US)

(72) Inventors: Maulik V. Shah, Scotch Plains, NJ (US); Ripudaman Singh, Hillsborough, NJ (US); Sami Ali, Jersey City, NJ (US); Janavananmay Panchal, Mumbai (IN)

(73) Assignee: MAXIS HEALTH LLC., Edison, CA (US)

(21) Appl. No.: 18/666,506

(22) Filed: May 16, 2024

(30) Foreign Application Priority Data
Sep. 29, 2023 (IN) 202341065604

(51) Int. Cl. A61B 5/16; A61B 5/00; G16H 10/20

(52) U.S. Cl. CPC A61B 5/165; A61B 5/6801; A61B 5/7264; A61B 5/744; A61B 5/746; A61B 5/7475; G16H 10/20

(57) ABSTRACT

The present invention relates an artificial intelligence (AI) based remote emotion detection and control system and method for detecting the symptoms of attention disorder, emotional dysregulation, mental stress, and impulsive dysregulation. The AI based remote emotion detection and control system comprises a wearable device and a computing device includes a guardian input module, a storage module, an assessment module, an alerting module, and an emotion module. The wearable device and the computing device are connected to a server via a network. The AI based remote emotion detection and control system provides quick and accurate care by detecting pre-atypical behaviour physiological patterns using the wearable device. The AI based remote emotion detection and control system improves in situ personalized care for people with autism, epilepsy, and other disorders that involve episodes of extreme emotional or physical responses.

### FIELD OF THE INVENTION

The present disclosure relates generally systems and methods for detecting abnormal mental and emotional health, and more particularly to an artificial intelligence (AI) based remote emotion detection and control system and method for detecting the symptoms of attention disorder, emotional dysregulation, mental stress, and impulsive dysregulation.

### BACKGROUND

Emotions are the instinctive state of mind of an individual that are catalyzed and brought on by neuropsychological changes. These changes can be triggered by a circumstance or in relation to others. When such changes take place, they almost instantaneously impact the physiological nature of a being. This complex amalgamation of consciousness, bodily sensations, and behaviour is often critical to one's wellbeing and holistic development.

Emotion dysregulation is a difficulty in managing one's emotions in a healthy way. Emotion dysregulation is expressed in the form of anger, frustration, and anxiety, which are all normal emotions but can become problematic when they are experienced too intensely or for too long. People with emotion dysregulation also have difficulty in identifying and expressing their emotions, or they resort to unhealthy coping mechanisms such as substance abuse or self-harm.

There are a number of possible causes of emotion dysregulation, including genetics, brain chemistry, and environmental factors such as childhood trauma. There is no one-size-fits-all treatment for emotion dysregulation, but there are a number of therapies that can be helpful, such as cognitive-behavioural therapy (CBT), dialectical behaviour therapy (DBT), and mindfulness-based therapies.

The brain undergoes a period of rapid development in the first few years of life. This is when neural connections are being formed at an astonishing rate. These connections are essential for learning, memory, and emotional regulation. Negative experiences, behaviours, or environmental factors can disrupt this development and lead to mental health problems. For example, children who experience abuse or neglect are more likely to develop anxiety, depression, and other mental health problems. Even relatively minor negative experiences, such as being teased or bullied, can have a lasting impact on a user's mental health. This is because these experiences can lead to feelings of insecurity, worthlessness, and fear.

Early experiences are not the only factor that determines a user's mental health. Genetics, family history, and other factors also play a role. However, early childhood experiences can have a profound impact on a user's development and mental health.

Disorders such as autism spectrum disorder (ASD), attention deficit hyperactivity disorder (ADHD), anxiety, depression, obsessive-compulsive disorder (OCD), oppositional defiant disorder (ODD), post-traumatic stress disorder (PTSD), and chronic stress can all alter a person's physiological baseline. As a result, it is critical to monitor and address even minor changes in these physiological bio-variables in order to assess a person's emotional and physical health.

There is no simple, objective test to determine whether an adult or a child has ADHD. However, a specialist can make a diagnosis after a detailed assessment. This assessment may include a physical examination, which can help rule out other possible causes of the symptoms. It may also include a series of interviews with the examinee, as well as interviews or reports from other significant people, such as partners, parents, and teachers. These methods rely heavily on the professionalism of the specialist and the cooperation of the examinee and the other significant people.

An attempt was made to diagnose schizophrenia using pre-pulse inhibition (PPI). PPI is a neurological phenomenon in which a weaker pre-stimulus (pre pulse) inhibits the reaction of an organism to a subsequent strong reflex-eliciting stimulus (pulse), often using the startle reflex. The stimuli are usually acoustic. The reduction or lack of reduction of the amplitude of the startle reflects the ability of the nervous system to temporarily adapt to a strong sensory stimulus when a preceding weaker signal is given to warn the organism (e.g., ears). In the test, known in the art as, the “San Diago” test, examinees were given acoustical stimulus and pre-stimulus and the startle response was measured using Electromyography (EMG). The test results were not conclusive and the method was abandoned.

Therefore, there is a need for an artificial intelligence (AI) based remote emotion detection and control system. There is a need for a method for detecting the symptoms of attention disorder, emotional dysregulation, and impulsive dysregulation. There is need for an AI based remote emotion detection and control system for detecting the symptoms of attention disorders, emotional dysregulation, and impulsive dysregulation independently, without the need of the professionalism of the specialist and/or the cooperation of the examinee. There is a need for an AI based remote emotion detection and control system that provides rapid evaluation and personalized intervention regimen for children, adults and old persons.

### SUMMARY OF THE INVENTION

The following presents a simplified summary of one or more embodiments of the present disclosure in order to provide a basic understanding of such embodiments. This summary is not an extensive overview of all contemplated embodiments, and is intended to neither identify key nor critical elements of all embodiments, nor delineate the scope of any or all embodiments.

The present disclosure, in one or more embodiments, relates to a remote emotion detection and control system. The remote emotion detection and control system comprises, a computing device, and a wearable device.

The wearable device is configured to collect at least one physiological parameter of a user or a user, such as a child or an adult or an old person, in real-time in the form of Sensor signals. The wearable device is configured with plurality of sensors for detecting the bio-signals. In specific, the bio-signals comprise, but are not limited to, volumetric changes in the blood flow, electrodermal activity (EDA), galvanic skin response (GSR), skin temperature (SKT), and body movement.

In specific, the sensors comprise, but are not limited to, a photoplethysmography (PPG), an electrodermal activity (EDA) sensor, an accelerometer (ACC), a skin temperature (SKT) sensor, galvanic skin response (GSR) sensor, inertial measurement unit (IMU), computer vision signals, and global positioning system (GPS). In one embodiment herein, the PPG is configured to monitor the heart rate of the user. In specific, the PPG is a simple sensor that uses a LED light source and a photodetector at the skin surface to detect volumetric changes in the blood flow. The EDA sensor is configured to measure and detect changes in electrical activity resulting from changes in sweat gland activity from the skin of the user. In specific, the EDA is also known as galvanic skin response (GSR). The ACC is configured to monitor the relative motion or physical activity of the user. The skin temperature sensor is configured to monitor the temperature of the user.

In one embodiment herein, the computing device having a controller and a memory for storing one or more instructions and plurality of modules executable by the controller. The wearable device and the computing device are in communication with a server via a network. The controller is configured to execute the one or more instructions to perform operations using the plurality of modules. The plurality of modules comprises a guardian input module, a storage module, an assessment module, an alerting module, and an emotion module.

The guardian input module is configured to provide a pre-assessment questionnaire to collect behaviour data of the user from at least one guardian in the form of responses and transmit the responses to the server. In specific, the guardian comprises either a parent or a caregiver or both the parent and the caregiver. The pre-assessment questionnaire is displayed on a user interface of the computing device of the guardian. The computing device comprises, but is not limited to, a smartphone, a laptop, a smart watch, and a computer.

The storage module is configured to store the physiological parameter of the user and a pre-atypical behaviour physiological pattern of the user triggered by the physiological parameter based on the responses entered by the guardian.

The assessment module is configured to analyze the physiological parameter and the responses of the guardian to detect a presence or an absence of the pre-atypical behaviour physiological pattern and thereby generate a user condition data. In specific, the user condition data is shared with at least one healthcare provider.

The alerting module is configured to transmit an alert to the guardian when the pre-atypical behaviour physiological pattern of the user is detected. In specific, the alert is in a form of a text or pop-up notification stating a suspected emotional dysregulation, or regulated states of the user.

The emotion module is configured to process the pre-atypical behaviour physiological pattern to recommend a personalized intervention regimen for the user based on the detected pre-atypical behaviour physiological pattern of the user. The personalized intervention regimen defines precautionary components.

In one embodiment herein, the personalized intervention regimen comprises just in time adaptive intervention (JITAI) such as Child intervention program (CIP), and parent coping program (PCP). In specific, the emotion module recommends personalized coping strategies based on the current emotion of the user and the guardian. The emotion module further assesses the emotion of the user and the guardian before providing step-by-step instructions to mitigate an ongoing emotion dysregulation episode of the user.

In specific, the emotion module derives the computer vision-based signals from the user's interactions with a digital avatar. The emotion module further modulates the personalized intervention regimen based on the computer vision-based signals by adjusting the personalized intervention regimen intensity accordingly.

One or more precautionary components are administered for the user through the user interface for remote emotional care according to the personalized intervention regimen generated for the user. The precautionary components comprise plurality of precautionary strategies such as the child intervention program (CIP) that includes biofeedback-based emotional rebalancing (BBER) with the digital avatar, guided breathing, and a heart rate variability (HRV) training instructed to the user on a routine basis.

According to another aspect, the invention provides a method for operating the remote emotion detection and control system. At first, the physiological parameter of the user is collected by the wearable device. In specific, the wearable device is configured to monitor the physiological parameter of the user in real-time. Next, the responses for the pre-assessment questionnaire are received from the guardian through the guardian input module for collecting behaviour data of the user. In specific, the responses are transmitted to the server.

Next, the physiological parameter of the user and a pre-atypical behaviour physiological pattern of the user triggered by the physiological parameter is stored in the storage module. Next, the physiological parameter and the responses are analysed by the assessment module to detect the presence or absence of the pre-atypical behaviour physiological pattern and thereby generate a user condition data report.

Next, the alert is transmitted to the guardian through the alerting module when the pre-atypical behaviour physiological pattern of the user is detected. Later, the pre-atypical behaviour physiological pattern is processed by the emotion module for recommending a personalized intervention regimen for the user based on the detected pre-atypical behaviour physiological pattern of the user.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

The accompanying drawings, which are incorporated in and constitute a part of the specification, illustrate an embodiment of the invention, and, together with the description, explain the principles of the invention.

FIG. 1 illustrates a perspective view of a block diagram of a remote emotion detection and control system, in accordance with embodiments of the invention.

FIG. 2 illustrates a flowchart of a method for operating the remote emotion detection and control system, in accordance with embodiments of the invention.

### DETAILED DESCRIPTION

Reference will now be made in detail to the present preferred embodiments of the invention, examples of which are illustrated in the accompanying drawings. Wherever possible, the same reference numerals are used in the drawings and the description to refer to the same or like parts.

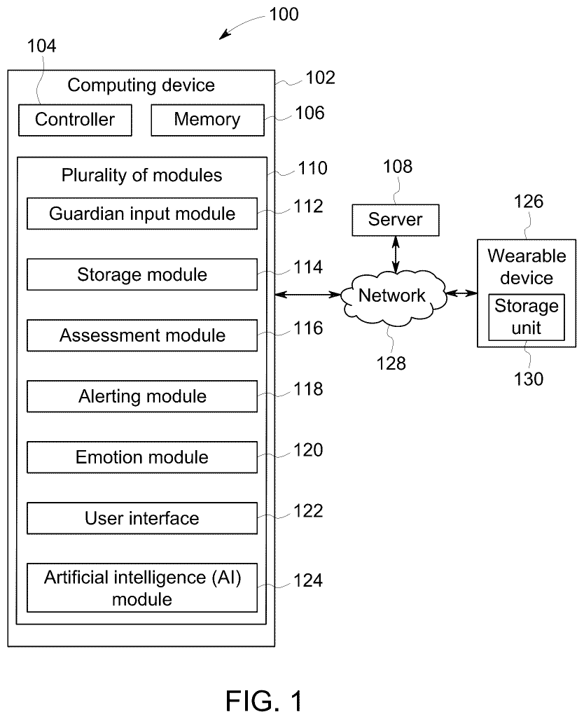

FIG. 1 refers to a perspective view of a block diagram of an artificial intelligence (AI) based remote emotion detection and control system 100. In one embodiment herein, the AI based remote emotion detection and control system 100 comprises, a computing device 102, and a wearable device 126.

The wearable device 126 is configured to collect at least one physiological parameter of a user or a user in real-time in the form of a bio-signal. At least four bio-signals are collected by the wearable device 126. The wearable device 126 is configured with plurality of sensors and a storage unit 130. In specific, the bio-signals comprise, but are not limited to, volumetric changes in the blood flow, an electrodermal activity (EDA) signal, a galvanic skin response (GSR) signal, a skin temperature (SKT) signal, an inertial measurement, computer vision signals, and body movement. The sensors comprise, but are not limited to, a photoplethysmography (PPG), an electrodermal activity (EDA) sensor, an accelerometer (ACC), a skin temperature (SKT) sensor, galvanic skin response (GSR) sensor, an inertial measurement unit (IMU), a computer vision unit, and global positioning system (GPS). The storage unit 130 is configured to store the Sensor-Signal detected by the sensors.

In an embodiment, the PPG is configured to monitor the heart rate of the user. In specific, the PPG is a simple sensor that uses a LED light source and a photodetector at the skin surface to detect volumetric changes in the blood flow. When the PPG transmits an infrared/green light through the skin, the blood absorbs the light. Based on the light reflection, variations in the blood flow is measured as changes in the light intensity. By processing this signal, the heart rate and the heart rate variability amongst other time, frequency and non-linear domain parameters are computed.

In an embodiment, the EDA sensor is configured to measure and detect changes in electrical activity resulting from changes in sweat gland activity from the skin of the user. In specific, the EDA is also known as galvanic skin response (GSR). The EDA refers to the variation of the electrical conductance of the skin in response to sweat secretion. By applying a low constant voltage to the skin, the variation in the skin conductance is measured by utilizing the electrical signal recorded by electrodes attached to the skin.

In an embodiment, the ACC is configured to monitor the relative motion or physical activity of the user. In specific, the ACC records the acceleration associated with body movement. The ACC is able to assess physical activity in three axes. The ACC uses an electromechanical sensor to capture the forces caused by the motion.

In an embodiment, the skin temperature sensor is configured to monitor the temperature of the user. In specific, the SKT is the temperature of the outermost surface of the body. The SKT is different from the core temperature. The SKT is a reliable indicator for assessing the healthy functions. The SKT is measured by infrared thermometers or thermistors. The computing device 102 having a controller 104 and a memory 106 for storing one or more instructions and plurality of modules 110 executable by the controller 104. The wearable device 126 and the computing device 102 are in communication with a server 108 via a network 128. The controller 104 is configured to execute the one or more instructions to perform operations using the plurality of modules 110. The plurality of modules 110 comprises a guardian input module 112, a storage module 114, an assessment module 116, an alerting module 118, and an emotion module 120.

The guardian input module 112 is configured to provide a pre-assessment questionnaire to collect behaviour data of the user from at least one guardian in the form of responses and transmit the responses to the server 108. In specific, the guardian comprises either a parent or a caregiver or both the parent and the caregiver. The pre-assessment questionnaire is displayed on a user interface 122 of the computing device 102 of the guardian. The computing device 102 comprises, but is not limited to, a smartphone, a laptop, a smart watch, and a computer.

The storage module 114 is configured to store the physiological parameter of the user and a pre-atypical behaviour physiological pattern of the user triggered by the physiological parameter based on the responses entered by the guardian. In an embodiment, the storage module 114 stores a database of past record of the user. The assessment module 116 is configured to analyze the physiological parameter and the responses of the guardian by using an artificial intelligence (AI) module 124 to detect a presence or an absence of the pre-atypical behaviour physiological pattern and thereby generate a user condition data.

In specific, the AI module 124 comprises multiple AI algorithms. In specific, the user condition data is shared with at least one healthcare provider based on the consent by the user or the guardian. The alerting module 118 is configured to transmit an alert to the guardian when the pre-atypical behaviour physiological pattern of the user is detected. In specific, the alert is in a form of a text or pop-up notification stating a suspected emotional dysregulation, or regulated states of the user.

The emotion module 120 is configured to process the pre-atypical behaviour physiological pattern to recommend a personalized intervention regimen for the user based on the detected pre-atypical behaviour physiological pattern of the user. The personalized intervention regimen defines precautionary components. In an embodiment, the personalized intervention regimen comprises just in time adaptive intervention (JITAI) such as child intervention program (CIP), and parent coping program (PCP). In specific, the emotion module 120 recommends personalized coping strategies based on the current emotion of the user and the guardian. The emotion module 120 further comprises episode debrief, digital health diary of the user.

In specific, the PCP is dynamically generated by the AI module 124, PCP recommends coping steps based on the severity of the detected pre-atypical behaviour physiological pattern and the past record of what worked and what didn't work in similar signals. Further, the PCP provides recommendation through a digital avatar that dynamically generates the coping strategy based on the AI algorithm of the AI module 124, which recommends the coping strategy based on severity of the detected signal as well as based on the past record of what worked and what did not work in similar signal. So, the PCP is highly personalized to specific signal at each occurrence and attuned to the situation in which the PCP is being recommended by AI to the guardian such as parents and caregivers.

In specific, the CIP is dynamically generated by the AI module 124, the CIP provides assistance through a digital avatar, the digital avatar proactively interprets the pre-atypical behaviour physiological pattern or mental stress directly from sensor signals, determines intervention, the user engagement strategy, synthesizes tone, interaction story, and facial expressions of the avatar interaction to respond in a more compassionate manner, and delivers intervention.

In one embodiment, the emotion module 120 derives the computer vision-based signals from the user's interactions with the digital avatar. In specific, the digital avatar is an AI-powered virtual avatar that is displayed in the wearable device 126. The emotion module 120 further modulates the personalized intervention regimen based on the computer vision-based signals by adjusting the personalized intervention regimen intensity accordingly.

The emotion module 120 further assesses the emotion of the user and the guardian before providing step-by-step instructions to mitigate an ongoing emotion dysregulation episode of the user. The emotion module 120 is responsible for analyzing emotions from the sensor Signals captured by the wearable device 126. The emotion module 120 identifies important features from each bio-signal. These features are then used by a machine learning algorithm of the AI module 124 to classify the emotion that the user is experiencing. The machine learning algorithm is any algorithm that is capable of classifying emotions, such as support vector machines, decision trees, or neural networks.

The AI module 124 is trained on a dataset of sensor Signals that have been labelled with the corresponding emotions. Once the AI module 124 is trained to classify the emotions of new sensor Signals. The emotion module 120 also includes a parameter resolution module and an individual baseline setting module. The parameter resolution module is responsible for determining the optimal values for the parameters of the machine learning algorithm. The individual baseline setting module is responsible for setting a baseline for each user's bio-signals. This baseline is used to account for individual differences in bio-signal data.

In one embodiment, the emotion module 120 comprises an AI-powered ecological momentary assessment (EMA) is state-sensitive and context-sensitive. The EMA is configured to detect changes in patterns, context, or sensor Signals of the user in real time. This allows the emotion module 120 to trigger frequent assessments and provide personalized, timely digital interventions for users' mental health. The emotion module 120 also utilizes digital phenotypes to correlate historic EMA data with relevant state or context data from the physiological parameter. This enables the emotion module 120 to detect symptoms early on, before the physiological parameter become more severe.

One or more precautionary components are administered for the user through the user interface 122 according to the personalized intervention regimen generated for the user. The precautionary components comprise plurality of precautionary strategies such as the child intervention program (CIP) that includes biofeedback-based emotional rebalancing (BBER) with the digital avatar, guided breathing, and a heart rate variability (HRV) training instructed to the user on a routine basis.

In an embodiment, the digital avatar is programmed to display an avatar that performs a reaction when the user's physiological pattern indicates that they are about to engage in pre-atypical behaviour physiological pattern. The reaction includes instructions to breathe deeply, biofeedback-based emotional rebalancing techniques, and guidance on how to step away from the situation.

In one embodiment, the assessment module 116 aids in detect emotions (the pre-atypical behaviour physiological pattern) based on the bio-signals. Thereby, the emotion module 120 verbalize the detected emotions through the digital avatar. The alerting module 118 transmits alerts to parents and caregivers in real time, creating a path for timely interventions for user who are unable to speak or express their emotions verbally with specificity. This feature is particularly beneficial for the user with disorders, which often have difficulty communicating their emotions. The emotion module 120 further aids parents and caregivers to better understand their child's needs and provide necessary support to the child.

In specific, the AI based remote emotion detection and control system 100 has the potential to make a significant impact on User with emotional dysregulation and communication disorder. By providing parents and caregivers with real-time insights into their child's emotional state, the AI based remote emotion detection and control system 100 aids them provide more effective support and interventions.

For instance, the digital avatar aids children with mental health challenges manage their emotions. The digital avatar detects emotional cues from Sensor Signals, assesses the need for intervention, plans an engagement strategy, and responds with compassion, mimicking the tone, storylines, and facial expressions of the child. This approach empowers children to engage in more natural self-interventions. The digital avatar offers verbal guidance through animations, crafts narratives and role-playing scenarios to aid children recognize and express their emotions, and promotes muscle relaxation.

In the AI based remote emotion detection and control system 100, the child interacts with the digital avatar that synthesizes emotions or mental stress directly from Sensor Signals. The AI algorithm of the AI module 124 decodes Sensor Signals into synthesized speech and facial expressions of any avatar, enabling the digital avatar to respond in a more compassionate manner to deliver intervention. This approach enables children or adults with mental health challenges to communicate and find ways to self-intervene more naturally.

The server 108 further includes a user database. In one embodiment user database includes a user profile and a pre-assessment data, the user condition data, a user personalized regimen data, and a user interaction data. The wearable device 126 can be any generic wearable device such as a smart watch, wristband, or body patch. The wearable device 126 uses the sensors to monitor the physiological parameters, such as heart rate, blood pressure, and body temperature, from either the user's wrist or another location on their body. The wearable device 126 collects data at a predetermined sampling frequency and sends collected data to the guardian, who can then cross-reference the data.

The precautionary components are administered to the user on a routine basis or administered as needed. The frequency and intensity of the interventions varies depending on the user's needs. The precautionary components are administered through the user interface 122 through the computing device 102. The user interface 122 is a web/mobile based platform that allows the user to access the precautionary strategies and to track their progress. The user interface 122 can also be used by caregivers to monitor the user's progress and to provide support. The precautionary components are an important part of the remote care. They can help to improve the user's emotional health and well-being, and they can help to reduce the risk of developing mental health problems.

Digital health diary syncs data with the emotion module 120, which activates the “Episode debrief” component. The digital health diary helps minimize recall bias in medical conditions, symptoms, and health trends. The digital health diary is shareable with healthcare providers in a convenient and editable form. These features make an AI based software a stand-out system for at least one of a remote paediatric care, an employee's mental state care and an emotional state of user, thereof.

The AI based software captures all incidences, regardless of whether the wearable device 126 is Worn or not Worn at the time or if a prediction is not made. This is because the AI based software also collects data from the digital health diary, which is a convenient and editable form of health information that can be shared with healthcare providers. The digital health diary helps minimize recall bias in medical conditions, symptoms, and health trends. This means that the AI based software is able to capture a more complete picture of the user's health, even if the wearable device 126 is not worn all the time. This increased data collection makes the personalized program even more robust. The program can be tailored to the user's needs, even if the user does not always wear the wearable device 126.

Further, the wearable device 126 generates unique digital phenotype from the user's continual usage. These digital phenotypes are unique benchmarks or baselines or contextual patterns of the individual's data. The data is synced through software to match patterns for personalized profiles. For example, the wearable device 126 may measure the user's heart rate, skin conductance, and breathing rate. The device can then generate the unique digital phenotype for each of these measurements. These digital phenotypes can be used to create a personalized profile for the user. The personalized profile is used to track the user's health and well-being. The wearable device 126 is use the personalized profile to provide the user with feedback and suggestions. For example, the wearable device 126 may alert the user if their heart rate is elevated or if their skin conductance is high. The wearable device 126 may also suggest exercises or relaxation techniques to help the user manage their stress levels.

In one embodiment, the assessment module 116 utilizes the AI algorithm to provide personalized suggestions based on ongoing monitoring data of the user and allows to integrate with Sensor Signals receive from the wearable device 126 of the user that are exhibiting emotional outbursts or stress. Then, the emotion module 120 translates the personalized suggestions that into the movements on the digital avatar's face, speech tone, story, and compassionate emotions making the digital avatar interact with the user to intervene with the facial movements for happiness, compassion and surprise thereof. The emotion module 120 verbalizes the emotions and creating a path for interventions in the user population that is unable to speak or express.

The emotion module 120 executes the AI algorithms in dynamic fashion. The emotion module 120 primary functions include the ongoing creation of the digital phenotypes and patterns, the development of personalized patterns, pattern matching, signal detection, recommendation generation, and data analysis. The emotion module 120 is trained on holistic real-world data and the emotion module 120 continue to develop robust and generalizable digital phenotypes, endpoints and biomarker to enhance personalization of targeted user's profile, anticipate health outcomes, and adapt the behaviour of avatars as needed.

In an embodiment, principal component analysis (PCA) is a dimensionality reduction technique that transforms a large set of features into a smaller one while preserving most of the information. Recursive feature elimination (RFE) is also used to find the best number of features. RFE is an algorithm that selects features by recursively removing the least important feature per iteration until the desired number of best features is reached. Finally, standardization is performed to prevent the machine learning classification model from over-fitting. Standardization is a process that brings all the features to a common scale without distorting the differences in the range of the values.

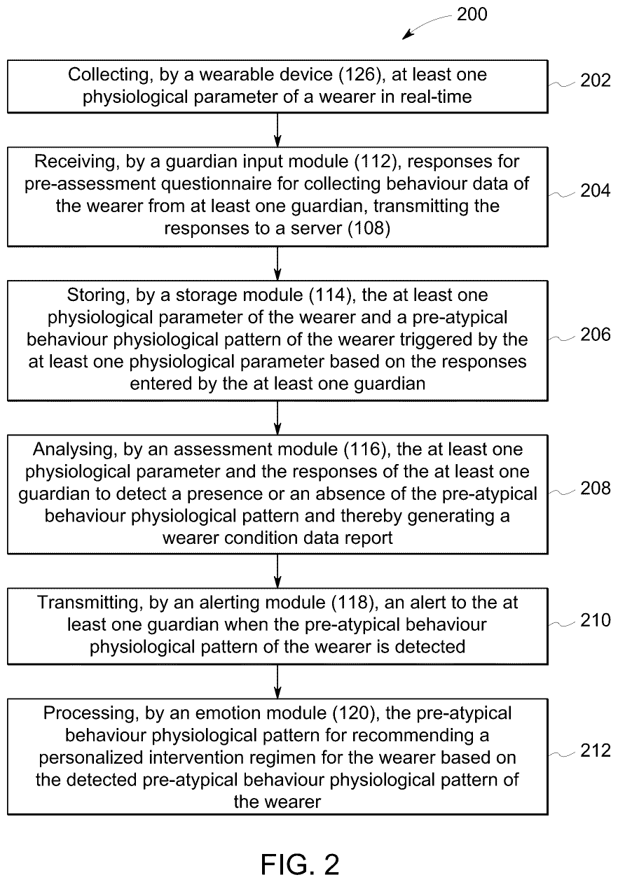

FIG. 2 refers to a flowchart 200 of a method for operating the AI based remote emotion detection and control system 100. At step 202, the physiological parameter of the user is collected by the wearable device 126. In specific, the wearable device 126 is configured to monitor the physiological parameter of the user in real-time. At step 204, the responses for the pre-assessment questionnaire are received from the guardian through the guardian input module 112 for collecting behaviour data of the user. In specific, the responses are transmitted to the server 108.

At step 206, the physiological parameter of the user and a pre-atypical behaviour physiological pattern of the user triggered by the physiological parameter is stored in the storage module 114. At step 208, the physiological parameter and the responses are analyzed by the assessment module 116 to detect the presence or absence of the pre-atypical behaviour physiological pattern and thereby generate a user condition data report. At step 210, the alert is transmitted to the guardian through the alerting module 118 when the pre-atypical behaviour physiological pattern of the user is detected. At step 212, the pre-atypical behaviour physiological pattern is processed by the emotion module 120 for recommending a personalized intervention regimen for the user based on the detected pre-atypical behaviour physiological pattern of the user.

For an instance, when the physiological parameter is detected by the wearable device 126. The responses are collected for in-the-moment questions by the guardian through the guardian input module 112. The guardian can use in-the-moment questions to get aids from a coach to optimize emotional equilibrium for the user, for example a child. By providing minimal information about the current scenario, the guardian can receive tailored strategies and tips to help the user in real time. Additionally, parents can take short training modules between emotion escalation episodes to learn new skills that can then be prompted during coaching. The in-the-moment questions are displayed in the form of multiple choice questions.

For example, can you do any of the following right now? Is any of the following happening for your child? For example, when your child doesn't feel well, they won't be able to make a plan to cope or solve the problem. They need a calmer and quieter environment. Select one or more of these steps to try such as Lower sound: Say less. Turn off TV or music, Lower light: Turn down lights, Lower touch: Give your child more space. Don't try to restrain or hold them, unless they want to be held, distract them: Offer a different activity, something to play with, or a snack.

Offer validation, remember your emotion coaching training module. Fill in the blanks to validate your child's feelings. “I can see you're feeling ______. That makes sense to me because ______” Remember, do not follow validation by saying “BUT” and starting to lecture!

Is my child trying to get a reaction from me? YES: [Coach on planned ignoring and then praising any behaviours toward calming down]. Tell your child, “Right now we're both getting more upset. I'm going to do something else until we can be calm”.

According to another exemplary embodiment of the invention, the AI based remote emotion detection and control system 100 a feature selection module (for each Sensor Signals captured), machine learning algorithm for emotion detection, analysis, parameter resolution and individual baseline setting.

In another embodiment, the AI-based remote emotion detection and control system 100 comprises three main components a training and testing module, a personalization component, and a monitoring component.

The training and testing module that is configured with machine learning algorithms for training the AI-based remote emotion detection and control system 100. The training and testing module is used to train the system to classify between normal and emotional dysregulated states. The training and testing module uses a variety of machine learning algorithms, such as support vector machines (SVM), k-nearest neighbours (KNN), and random forests. The best algorithm is selected based on its performance on a test set.

The personalization component that receives feedback from the user to improve the performance of the AI-based remote emotion detection and control system 100. The personalization component receives feedback from the user about the system's predictions. This feedback is used to improve the system's accuracy by identifying patterns that the AI-based remote emotion detection and control system 100 has not yet learned.

The monitoring component that continuously monitors the user's biometrics and predicts when they are experiencing negative emotions. The monitoring component continuously monitors the user's biometrics, such as heart rate, skin conductance, and breathing rate. The AI-based remote emotion detection and control system 100 uses these biometrics to predict when the user is experiencing negative emotions, such as anxiety or stress.

If the AI-based remote emotion detection and control system 100 predicts that the user is experiencing a negative emotion, the AI-based remote emotion detection and control system 100 will send an alert to the user or their caregiver. The AI-based remote emotion detection and control system 100 is a personalized system that is able to learn the user's unique patterns of emotions. This allows the AI-based remote emotion detection and control system 100 to provide more accurate predictions and improve the user's overall well-being.

The AI-based remote emotion detection and control system 100 is a powerful tool that aids people manage their emotions and improve their overall well-being. The AI-based remote emotion detection and control system 100 is still under development, but it has the potential to revolutionize the way we think about mental health.

In one embodiment herein, the AI based remote emotion detection and control system 100 and method for detecting the symptoms of attention disorder, emotional dysregulation, and impulsive dysregulation by detecting responses. The AI based remote emotion detection and control system 100 provides quick and accurate remote paediatric care. The system 100 detects a pre-atypical behaviour physiological pattern by using the wearable device 126. The system 100 improves in situ personalized care to people with autism, epilepsy, and other disorders that involve episodes of extreme emotional or physical responses. The system 100 monitors the physiological symptoms would capture the impact of psychological sources of stress.

The system 100 accurately detecting the symptoms of attention disorders, emotional dysregulation, and impulsive dysregulation independently, without the need of the professionalism of the specialist and/or the cooperation of the examinee. The system 100 can detecting the symptoms of attention disorders, emotional dysregulation, and impulsive dysregulation from home, during commute, or otherwise away from the office. The artificial intelligence based system 100 for emotional dysregulation assessment and care continuum provides a comprehensive approach for assessment of emotion and provides complete care continuum to the user. The predictive, proactive and comprehensive emotion management system 100 provides personal emotion mentoring to guide the user and remind the parents of consultations.

In an embodiment, the AI based remote emotion detection and control system 100 can help employers (not limited to stockbroker/fire fighter/Veterans) monitor employee stress levels and identify areas where professional training or support may be beneficial. By offering biofeedback sessions, employers can help employees reduce stress and improve productivity. This can lead to decreased absenteeism and increased profits.

The AI based remote emotion detection and control system 100 is more patient-centric, thereby helping pharmaceutical and life sciences clinical trials' sponsor improve their chances of success in clinical development as well as improve outcome by enhancing patient engagement and retention in clinical trials. The AI based remote emotion detection and control system 100 uses wearable data to deliver feedback based on expressed emotions. The AI based remote emotion detection and control system 100 translate the raw data from wearable device 126 into actionable emotional insights, which can be used to improve patient experiences throughout the clinical trial process; to understand patient preferences expressed via emotional responses at every stage in clinical trials and by leveraging that into improving study design, to enhance patient engagement and retention in clinical trials; to drive more patient-centric clinical trials with an improved outcome. The AI based remote emotion detection and control system 100 conducts a comprehensive analysis of content performance and identifies the types of experiences that effectively drive customer engagement.

The AI based remote emotion detection and control system 100 continuously monitors user's biofeedback while they are viewing or listening to the digital content. The system 100 detects user's emotional responses and attention to the content in real-time. This information is then sent back to digital content streaming platforms, which can use it to enhance, change, shorten, create automated durations, recommend, or end specific content. This can help to improve consumer retention, personalization, and commercial outcomes by making digital streaming more connected with consumers and personalized based on their emotional responses to the content in real-time.

The AI based remote emotion detection and control system 100 analyses how consumers interact with content to determine what types of experiences effectively drive engagement. The system 100 also understands how consumers' emotional state can influence their engagement, preference, and loyalty. This information is used to determine the appropriate types of content to deliver and for how long, with the goal of maximizing retention and the consumer experience.

By using this feature, the AI based remote emotion detection and control system 100 allows businesses to improve their digital content strategies and increase customer retention rates. The system 100 ensures that messages are delivered in a timely and relevant manner and allows for the customization of push notifications based on specific emotional triggers. The system 100 uses a defined logic to determine when and how to send push notifications based on these triggers, while also considering individual delivery preferences. Through performance analysis, the system 100 can understand the emotional impact of push notifications across different customer segments, which helps to identify key trends for message optimization. The system 100 also enhances quality monitoring and makes it completely consumer centric.

User's wearable devices will be connected to the AI based remote emotion detection and control system 100 through at least one of a software application or an AI based application. The AI based application will send insights to digital content providers' software applications in real-time, based on the consumer's prior consent and authorization. The users include children, parents, and old people. This will allow digital content providers to leverage insights generated in real-time from the consumer's content experience, and then perform a variety of personalization actions to improve the consumer's experience.

In an embodiment herein, the computing device 102 is at least one of, but not limited to, a smartphone, a mobile phone, a tablet, a dedicated device, and a personal digital assistant (PDA). In an embodiment herein, the computing device 102 includes an AI based web application running but not just limited to within a cloud server. In some embodiments, the computing device 102 includes a dedicated AI based mobile application. The computing device 102 includes an operating system that coordinates the use of hardware and software resources, as well as one or more applications (for example, web browser, AI based web application, and dedicated AI based mobile applications).

The AI based remote emotion detection and control system 100 ensures the delivery of timely and relevant messages, allowing for the customization of push notifications based on specific emotional triggers. The AI based remote emotion detection and control system 100 defined logic to determine when and how to send push notifications based on these triggers, while also accommodating individual delivery preferences. Through performance analysis. The AI based remote emotion detection and control system 100 further enhances quality monitoring and makes it completely consumer centric.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principles of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

It will readily be apparent that numerous modifications and alterations can be made to the processes described in the foregoing examples without departing from the principles underlying the invention, and all such modifications and alterations are intended to be embraced by this application.

The claimed invention is:

1. An artificial intelligence (AI) based remote emotion detection and control system, comprising:
a wearable device adapted to be worn by a user, wherein the wearable device comprises plurality of sensors that is configured to detect at least one physiological parameter of the user;
a computing device having a controller and a memory for storing one or more instructions and plurality of modules executable by the controller,
wherein
the wearable device and the computing device are in communication with a server via a network,
the controller is configured to execute the one or more instructions to perform operations using the plurality of modules,
wherein the plurality of modules comprises:
a guardian input module configured to provide a pre-assessment questionnaire to collect behaviour data of the user from at least one guardian in form of responses, wherein the responses comprises a pre-atypical behaviour physiological pattern of the user;
a storage module configured to store the at least one physiological parameter of the user and the pre-atypical behaviour physiological pattern of the user triggered by the at least one physiological parameter based on the responses entered by the at least one guardian;
an assessment module configured to analyze the at least one physiological parameter and the responses of the at least one guardian to detect a presence of the pre-atypical behaviour physiological pattern using an artificial intelligence (AI) module, thereby generating a user condition data report;
an alerting module configured to transmit one or more alerts to the at least one guardian when the pre-atypical behaviour physiological pattern of the user is detected; and
an emotion module configured to process the detected pre-atypical behaviour physiological pattern to recommend a personalized intervention 5 regimen for the user by using the artificial intelligence (AI) module through a digital avatar, wherein the personalized intervention regimen defines precautionary components,
whereby the AI based remote emotion detection and control system detects and verbalizes the pre-atypical behaviour physiological pattern of the user through the digital avatar and transmits alerts to the at least one guardian in real time, creating a path for timely interventions in the user.

2. The AI based remote emotion detection and control system of claim 1, wherein said plurality of sensors comprises at least one of a photoplethysmography (PPG), an electrodermal activity (EDA) sensor, an accelerometer (ACC), a skin temperature (SKT) sensor, inertial measurement unit (IMU), global positioning system (GPS), computer vision signals.

3. The AI based remote emotion detection and control system of claim 2, wherein said PPG is configured to monitor heart rate of the user, wherein the EDA is configured to measures and detect changes in electrical activity resulting from changes in sweat gland activity from the skin of the user, wherein the ACC is configured to monitor relative motion or physical activity of the user, wherein the SKT is configured to monitor temperature of the user.

4. The AI based remote emotion detection and control system of claim 1, wherein said emotion module derives the computer vision-based signals from the user's interactions with the digital avatar, wherein the emotion module further modulates the personalized intervention regimen based on the computer vision-based signals by adjusting the personalized intervention regimen intensity accordingly.

5. The AI based remote emotion detection and control system of claim 1, wherein said pre-assessment questionnaire is display on a user interface of the computing device, wherein the computing device comprises at least one of a smartphone, a laptop, smart-watch, and a computer.

6. The AI based remote emotion detection and control system of claim 1, wherein said alert is in a form of a text or a pop-up notification stating the detection of the pre-atypical behaviour physiological pattern comprises at least one of an emotional dysregulation, post-traumatic stress disorder, obsessive compulsive disorder, attention deficit hyperactivity disorder, autism spectrum disorder, and oppositional defiant.

7. The AI based remote emotion detection and control system of claim 1, wherein said emotion module recommends personalized coping strategies based on current emotion of the user and the at least one guardian, wherein the emotion module further assesses the current emotion of the user and the at least one guardian before providing step-by-step instructions to mitigate an ongoing pre-atypical behaviour physiological pattern of the user.

8. The AI based remote emotion detection and control system of claim 1, wherein said one or more precautionary components are administered through the user interface for remote emotional care according to the personalized intervention regimen generated for the user, wherein the one or more precautionary components comprises plurality of precautionary strategies such as biofeedback-based emotional rebalancing (BBER), guided breathing sessions, heart rate variability (HRV) training instructed to the user on a routine basis through the digital avatar.

9. The AI based remote emotion detection and control system of claim 1, wherein said AI based remote emotion detection and control system detects users' emotional response and attention to digital content in real-time by continuous monitoring of users biofeedback while they are experiencing or engaging into any digital content by viewing or listening to the digital content.

10. A method for operating an artificial intelligence (AI) based remote emotion detection and control system, comprising:
collecting, by a wearable device, at least one physiological parameter of a user in real-time;
receiving, by a guardian input module, responses for pre-assessment questionnaire for collecting behaviour data of the user from the guardian, transmitting the responses to a server;
storing, by a storage module, the at least one physiological parameter of the user and a pre-atypical behaviour physiological pattern of the user triggered by the at least one physiological parameter based on the responses entered by the at least one guardian;
analysing, by an assessment module, the at least one physiological parameter and the responses of the at least one guardian to detect a presence or an absence of the preatypical behaviour physiological pattern and thereby generating a user condition data report;
transmitting, by an alerting module, an alert to the at least one guardian when the pre-atypical behaviour physiological pattern of the user is detected; and
processing, by an emotion module, the pre-atypical behaviour physiological pattern for recommending a personalized intervention regimen for the user based on the detected pre-atypical behaviour physiological pattern of the user.
